(19) United States
(12) Patent Application Publication
(10) Pub. No.: US 2024/0293571 A1
(43) Pub. Date: Sep. 5, 2024

(54) BRAIN-TARGETED ANTIBODY NANOPARTICLE FOR NEURODEGENERATIVE DISEASES THERAPY

(71) Applicant: UNIVERSITY OF SOUTH CAROLINA, COLUMBIA, SC (US)

(72) Inventors: PEISHENG XU, CHAPIN, SC (US); MINGMING WANG, WEST COLUMBIA, SC (US)

(21) Appl. No.: 18/559,145

(22) PCT Filed: Mar. 3, 2022
(86) PCT No.: PCT/US2022/018696

§ 371 Date: Nov. 6, 2023

Related U.S. Application Data
(60) Provisional application No. 63/230,965, filed on Aug. 9, 2021.
(60) Provisional application No. 63/185,157, filed on May 6, 2021.

(51) Int. Cl. A61K 47/69; A61K 9/00; A61K 31/197; A61K 47/60; A61P 25/28

(52) U.S. Cl. CPC A61K 47/6903; A61K 9/0019; A61K 31/197; A61K 47/60; A61P 25/28

(57) ABSTRACT

Disclosed are brain-targeted antibody delivery systems, methods of forming the systems, and methods of using the systems. The systems include hydrophilic nanogels based upon poly(ethylene glycol) copolymers. The nanogels can encapsulate an antibody for delivery to the brain and can include ligands for blood brain barrier (BBB) receptors on the surface, and as such, can utilize a systemic route of administration for delivery of the antibody to the brain and the eye. The systems can be utilized for systemic delivery routes to deliver the antibody to the brain side of the blood brain barrier and inside glial cells and degrade its corresponding protein. The systems can be utilized in treatment of neurodegenerative or retinal disorders.

### CROSS REFERENCE TO RELATED APPLICATION

This application, filed under the Patent Cooperation Treaty, claims filing benefit of U.S. Provisional Patent Application Ser. No. 63/185,157, having a filing date of May 6, 2021, and of U.S. Provisional Patent Application Ser. No. 63/230,965, having a filing date of Aug. 9, 2021, both of which are incorporated herein by reference for all purposes.

### STATEMENT REGARDING FEDERALLY SPONSORED RESEARCH OR DEVELOPMENT

This invention was made with government support under Grant No. 1 R01AG054839-01A1, awarded by the National Institutes of Health. The government has certain rights in the invention.

### BACKGROUND

Neurodegenerative diseases, including Alzheimer's diseases, Parkinson's disease, multiple sclerosis, neonatal hypoxic-ischemic, stroke, Amyotrophic lateral sclerosis, Huntington's disease, spinal cord injury, brain injury, retina injury, post-traumatic stress disorder, frontotemporal dementia, etc. are associated with the death of neurons caused by toxic substances and/or inflammatory microenvironment in the brain.

Traditional approaches for the treatment of such diseases are focused on reducing the amount of the toxic substances and attenuating the inflammatory status of the brain. Several studies have been carried out that achieved plausible results investigating the possibility of regenerating the missing neurons with local injection (intracerebroventricular, intravitreal, and intranigral) of therapeutics. However, due to the existence of the blood brain barrier, none of the studies managed to attain rescue of function of the brain by regenerating the lost neurons through systemic administration.

What is needed in the art are brain-targeted antibody delivery systems. For instance, brain-targeted systems that can deliver across the blood brain barrier antibodies that can encourage conversion of astrocytes into neurons via a systemic route of administration would be of great benefit in the art.

### SUMMARY

According to one embodiment, disclosed is a method for treating a neurodegenerative or retinal disorder. The method can include systemic delivery of a composition including a nanogel to a subject. The nanogel includes a crosslinked network including a first copolymer and a second copolymer. A first copolymer can include a first polymer backbone, a first group pendant to the first backbone that includes a first poly(ethylene glycol), and a second group pendant to the first backbone that is conjugated to an antibody. A second copolymer can include a second polymer backbone, a third group pendant to the second backbone that includes a second poly(ethylene glycol), and a fourth group pendant to the second backbone that comprises a phosphorylcholine group. A nanogel can also include a ligand for a blood brain barrier (BBB) receptor present at the surface of the nanogel that can be conjugated to additional pendant groups of the first and/or second copolymer. Following the systemic delivery, the ligand for the BBB receptor and the phosphorylcholine group can facilitate crossing of the BBB by the nanogel and can further facilitate uptake of the nanogel by cells, e.g., glial cells. The crosslinked network of the nanogel can degrade within the environment encountered by the nanogel following crossing of the BBB, upon which the antibody carried by the nanogel can be released from the first copolymer. The antibody can be configured to target an antigen on the brain side of the BBB, e.g., within glial cells, to provide a desired treatment following release, e.g., within the subject's brain or eyes. For instance, a phosphorylcholine group can function as a ligand for a nicotinic acetylcholine receptor (nAchR) and the antibody can include an anti-polypyrimidine tract binding protein 1 (anti-PTB-P1).

Also disclosed is a nanogel that includes a crosslinked network including first and second copolymers. A first copolymer can include a first polymer backbone, a first group pendant to the first backbone that includes a first poly(ethylene glycol), and a second group pendant to the first backbone that is conjugated to an antibody. A second copolymer can include a second polymer backbone, a third group pendant to the second backbone that includes a second poly(ethylene glycol), and a fourth group pendant to the second backbone that comprises a phosphorylcholine group. A nanogel can also include a ligand for a BBB receptor present at the surface of the nanogel. The BBB receptor ligand can be conjugated to additional pendant groups of the first and/or second copolymer. Each of the first and second copolymers can include a disulfide linkage and an ester linkage between the polymer backbone and the antibody, respectively.

Also disclosed is a method for forming a nanogel. For instance, a method can include forming a first copolymer by a conjugation of a first precursor copolymer with an antibody. The first precursor copolymer can include a backbone, first groups pendant to the backbone that include a first poly(ethylene glycol), second groups pendant to the backbone that include a terminal pyridine group, and third groups pendant to the backbone that are activated and terminate in a functional group (e.g., a nitrophenyl carbonate) that reacts with the antibody so as to conjugate the antibody to the polymer.

The method can also include forming a second copolymer by reaction of a second precursor copolymer with a monomer that includes a phosphorylcholine group. The second precursor polymer can include a backbone, first groups pendant to the backbone that include a second poly(ethylene glycol), and second groups pendant to the backbone that include a terminal pyridine. The reaction can include reaction of a portion of the terminal pyridine groups with the monomer and the resulting second copolymer can thus include a backbone, first groups pendant to the backbone that include the second poly(ethylene glycol), second groups pendant to the backbone that include unreacted pyridine groups, and third groups pendant to the backbone that include the phosphorylcholine group.

The nanogel can be formed upon crosslinking of the first copolymer and the second copolymer. The nanogel resulting from the crosslinking reaction of the first and second copolymers can include hydrophilic poly(ethylene glycol) groups at a surface of the nanogel, phosphorylcholine groups at a surface of the nanogel, unreacted pyridine groups at a surface of the nanogel and the antibody encapsulated within the crosslinked nanogel. Reaction of one or both of the nanogel copolymers with a ligand for a BBB receptor can serve to conjugate the BBB receptor ligand to the nanogel via reaction of the ligand with unreacted groups of the copolymer(s). In some embodiments, conjugation of the ligand for the BBB receptor can be carried out following crosslinking and formation of the nanogel, so as to ensure high concentration of the BBB receptor ligand at the surface of the nanogel.

In some embodiments, a formation method can include formation of one or both of the precursor copolymers by initial reaction of a poly(ethylene glycol) with a pyridine-2-thiol-containing monomer to form a precursor copolymer or to form an intermediate copolymer. In those embodiments in which the initial reaction forms an intermediate copolymer, further reaction(s) of the intermediate copolymer with one or more additional monomers can be carried out to form the precursor copolymer.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 presents a reaction scheme for formation of poly[(2-(pyridin-2-yldisulfanyl)ethyl acrylate)-co-[poly(ethylene glycol) methoxy]] (PDA-PEG-OCH₃).

FIG. 2 presents a reaction scheme for formation of poly[(2-(pyridin-2-yldisulfanyl)ethyl acrylate)-co-[poly(ethylene glycol) hydroxy]] (PDA-PEG-OH).

FIG. 3 presents a reaction scheme for formation of poly[(2-(pyridin-2-yldisulfanyl)ethyl acrylate)-co-[poly(ethylene glycol)[-co-[poly(2-mercaptoethanol)]](PDA-PEG-BME) intermediate copolymer.

FIG. 4 presents a reaction scheme for formation of poly[(2-(pyridin-2-yldisulfanyl)ethyl acrylate)-co-[poly(ethylene glycol)[-co-[poly(4-nitrophynyl chloroformate)]] (PDA-PEG-NPC) precursor copolymer.

FIG. 5 presents a reaction scheme for formation of poly[(2-(pyridin-2-yldisulfanyl)ethyl acrylate)-co-[poly(ethylene glycol)]-co-[poly(glutathione-2-methacryloyloxyethyl phosphorylcholine)]] (PDA-PEG-MPC) copolymer.

FIG. 6 presents a reaction scheme for formation of a PDA-PEG-antibody conjugate copolymer.

FIG. 7 presents a reaction scheme for formation of a PDA-PEG-MPC-antibody nanogel through crosslinking of the PDA-PEG-MPC copolymer with the PDA-PEG-antibody conjugate copolymer.

FIG. 8 presents a reaction scheme for formation of a dual targeted nanogel through surface decoration of a nanogel with a ligand for a BBB receptor.

FIG. 9 compares immunostaining results of cells treated with an antibody alone or with an antibody conjugated nanogel as disclosed herein.

FIG. 10 illustrates results of a nest construction assay for mice following treatment with either an antibody alone or with an antibody conjugated nanogel as disclosed herein.

FIG. 11 illustrates behavior evaluations through a Morris water maze (MWM) test.

FIG. 12 graphically presents behavior evaluation through the MWM test.

FIG. 13 graphically presents behavior evaluation through the MWM test.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

The present disclosure is generally directed to brain-targeted antibody delivery systems, methods of use thereof, and methods of forming the systems. Beneficially, disclosed systems can cross the blood brain barrier (BBB), and as such, can utilize a systemic route of administration for delivery of an antibody to the brain or the eyes, which makes in vivo application of the technology both practical and feasible.

In one embodiment, the system can carry an antibody that can induce the degrading of a specific endogenous protein, for instance, so as to convert astrocytes into neurons in the brain. However, it will be understood by one of skill in the art that disclosed delivery systems can be utilized to carry antibodies that can provide a useful function other than, or in addition to, those specifically referenced herein. In one embodiment, disclosed delivery systems can include an antibody that can be used for the treatment of neurodegenerative or retinal degenerative diseases, including and without limitation to, Alzheimer's disease, Amyotrophic lateral sclerosis, Friedreich's ataxia, Huntington's disease, Lewy body disease, Parkinson's disease, spinal muscular atrophy, multiple sclerosis, neonatal hypoxic-ischemic, stroke, spinal cord injury, brain injury, retina injury, age-related macular degeneration, myopic related macular degeneration, post-traumatic stress disorder, and frontotemporal dementia.

Disclosed delivery systems can be in the form of nanogel particles that include crosslinked copolymers that provide the nanoparticle structure of the delivery system, as well as that carry desired functional components (antibodies, BBB receptor ligands, targeting ligands, etc.). For instance, copolymers of a crosslinked nanogel can be conjugated with one or more of an antibody, a ligand for a BBB receptor, and/or one or more additional desirable components such as ligands for targeting a particular cellular component.

As utilized herein, the term “nanogel” refers to a particle having an average cross-sectional dimension on the nanoscale, e.g., less than 1000 nanometers, such as about 500 nanometers or less, about 300 nanometers or less, about 100 nanometers or less, about 50 nanometers or less, about 30 nanometers or less, about 20 nanometers, or about 10 nanometers or less, such as from about 5 nanometer to about 50 nanometers in some embodiments. A nanogel particle can also be highly hydrophilic and capable of high water content.

A copolymer of a crosslinked nanogel can be developed from an initial reaction product of a poly(ethylene glycol) and a pyridine-2-thiol-containing monomer. A copolymer can be a block copolymer, a random copolymer, or any combination thereof. While illustrated in the present disclosure as block copolymers, it will be understood that this representation is simply shorthand for any type of copolymer (e.g., random, block, etc.) that includes repeating units, including pendant groups as described herein.

A copolymer formed upon reaction of a poly(ethylene glycol) and a pyridine-2-thiol-containing monomer can include first groups pendant to the polymer backbone that include hydrophilic poly(ethylene glycol), as well as second groups pendant to the polymer backbone that are pyridine-terminated and that also include a disulfide linkage between the polymer backbone and the terminal pyridine group. Pyridine groups of a copolymer thus formed can be further reacted in one or more intermediate formation steps to conjugate desirable components of a delivery system to the copolymer and form copolymers that can then be crosslinked to form a nanogel.

For instance, a first copolymer can include an antibody conjugated to a pendant group and a second copolymer can include a phosphorylcholine group as a component of the pendant group (e.g., a terminal component of a pendant group), and both copolymers can include hydrophilic poly(ethylene glycol) pendant groups as well as unreacted groups for further conjugation. Upon crosslinking among and between the two (or more) copolymers, e.g., a crosslinking reaction in which disulfide bonds of unreacted pendant groups of the copolymers are cleaved followed by aerial oxidation, a resulting nanogel particle can be formed that can encapsulate the antibody as well as display hydrophilic poly(ethylene glycol) and other desirable groups at the surface and/or encapsulated within the nanogel. A ligand for a BBB receptor can also be conjugated to a copolymer so as to be present on the external surface of the crosslinked nanogel and encourage transport of the nanogel across the BBB. For instance, a ligand for a BBB receptor can be conjugated to remaining reactive groups of one or more of the copolymers following crosslinking and nanogel formation.

The nanogel particles thus formed can be suitable for safe and effective therapy and can include a hydrophilic poly(ethylene glycol) component, a phosphorylcholine component, a BBB receptor ligand, and an antibody for therapeutic delivery. The BBB receptor ligand can be present in high concentration at the exterior surface of the nanogel particles. The formation of the nanogel particles can endow advantages for central nervous system therapy. For example, due to the existence of the hydrophilic poly(ethylene glycol) pendant groups, the circulation time of the nanogels in a systemic delivery approach can be greatly extended. Moreover, the antibody to be delivered by the system can be encapsulated within the crosslinked nanogels and thus protected from degradation prior to delivery from the nanogel following crossing of the BBB. As the copolymers forming the nanogels and conjugating the antibody to the copolymers can include linkages sensitive to an environment common on the brain side of the BBB barrier, an antibody can be released from the nanogels upon passage of the nanogel into an environment conducive to linkage degradation, e.g., following crossing of the BBB by the nanogel and in some embodiments following uptake by cells on the brain side of the BBB.

A poly(ethylene glycol) used in forming a delivery system can include reactivity for reaction with a pyridine-2-thiol monomer, e.g., a methacrylate functionality. In one particular embodiment, an initial copolymer formation scheme can include reaction of pyridine-2-thiol monomer with poly(ethylene glycol) methacrylate having the general structure:

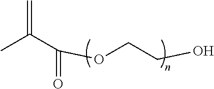

in which n is from about 4 to about 1,000, from about 5 to about 100, or from about 6 to about 20 in some embodiments.

A poly(ethylene glycol) methacrylate is not limited to the above, however, and can include modification at a terminal hydroxyl group. For instance, the poly(ethylene glycol) starting material can include, without limitation, poly(ethylene glycol) methacrylate, poly(ethylene glycol) methyl ether methacrylate, etc.

A pyridine-2-thiol-containing monomer can be copolymerized with a poly(ethylene glycol) to form a copolymer that includes the pyridine-2-thiol groups pendant to the copolymer backbone. By way of example, and without limitation, pyridine-2-thiol monomers can include one or more of:

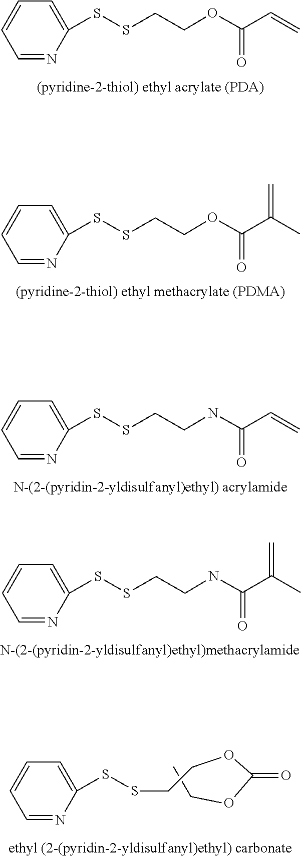 [SMILES: C=CC(=O)OCCSSc1ccccn1 ; C=C(C)C(=O)OCCSSc1ccccn1 ; C=CC(=O)NCCSSc1ccccn1 ; C=C(C)C(=O)NCCSSc1ccccn1 ; CCOC(=O)OCCSSc1ccccn1]

The reaction can be facilitated by any suitable catalyst. For example, a catalyst can include, without limitation, azobisisobutyronitrile (AIBN), benzoyl peroxide, potassium persulfate, or combinations thereof. The polymerization can be free radical polymerization or living radical polymerization including stable free radical mediated polymerization (SFRP), atom transfer radical polymerization (ATRP), reversible addition-fragmentation chain transfer (RAFT) polymerization, and iodine-transfer polymerization. The last monomer of the above examples (ethyl(2-(pyridin-2-yldisulfanyl)ethyl) carbonate) can be polymerized using isopropanol as an initiator and Sn(Oct)₂ as a catalyst through ring-opening polymerization.

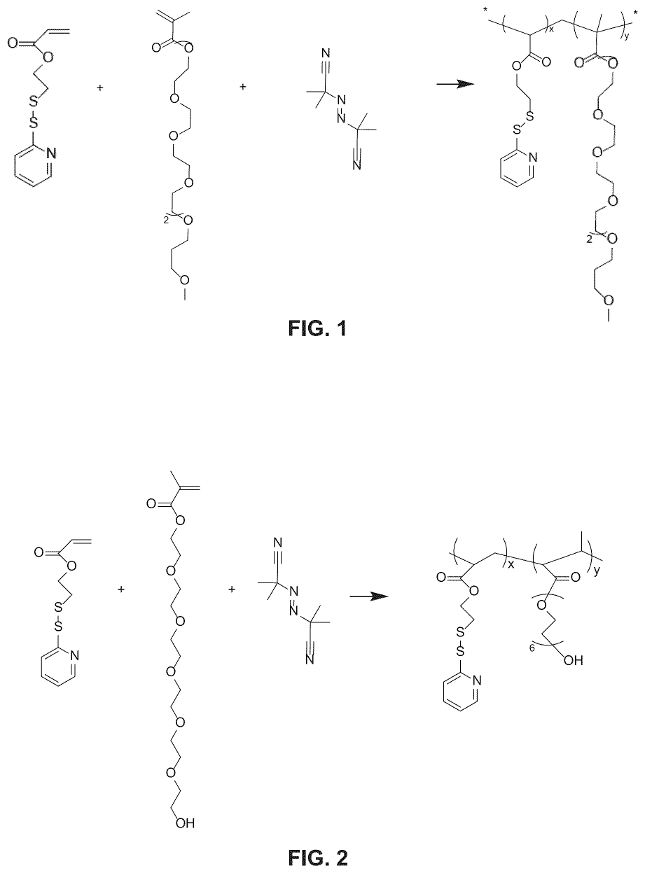

By way of example, FIG. 1 illustrates a reaction scheme of a 2-(pyridin-2-yldisulfanyl)ethyl acrylate (PDSA) monomer with poly(ethylene glycol) methyl ether methacrylate (PEGMMA), and FIG. 2 illustrates a reaction scheme of PDSA with poly(ethylene glycol) methacrylate (PGMA), both using azobisisobutyronitrile (AIBN) as catalyst. As can be seen, the poly(ethylene glycol) component of the copolymers thus formed can be in the form of groups pendant to the copolymer backbone, with second pendant groups including a disulfide linkage and an ester linkage and terminating in a pyridine group.

The molar ratio of the pyridine-2-thiol-containing repeating units of the polymer to the poly(ethylene glycol) pendant units can be from about 100:1 to about 1:100 (the ratio of x:y in FIG. 1 and FIG. 2), for instance, from about 20:1 to about 1:20 in some embodiments, from about 10:1 to about 1:10 in some embodiments, or about 1:1 in some embodiments.

A copolymer can generally have a weight average molecular weight from about 1,000 to about 100,000 or from about 5,000 to about 35,000 in some embodiments. In one embodiment, the copolymer can have a polydispersity index (PDI) of from about 1.05 to about 3 or from about 1.15 to about 1.30, in some embodiments.

The PDI is a measure of the distribution of molecular mass in a given polymer sample. The PDI calculated is the weight average molecular weight divided by the number average molecular weight. It indicates the distribution of individual molecular masses in a batch of polymers. The PDI has a value equal to or greater than 1, but as the polymer chains approach uniform chain length, the PDI approaches unity (i.e., 1).

The number average molecular weight (Mn) is readily calculated by one of ordinary skill in the art, and generally refers to the ordinary arithmetic mean or average of the molecular weights of the individual macromolecules. It is determined by measuring the molecular weight of n polymer molecules, summing the weights, and dividing by n, such as represented in the formula:

$${\overline{M}}_{n} = \frac{{\sum }_{i} {N}_{i} {M}_{i}}{{\sum }_{i} {N}_{i}}$$

where Nᵢ is the number of molecules of molecular weight Mᵢ. The number average molecular weight of a polymer can be determined by gel permeation chromatography, and all colligative methods, like vapor pressure osmometry or end-group determination.

The weight average molecular weight (M_{w}) is readily calculated by one of ordinary skill in the art, and generally refers to:

$${\overline{M}}_{w} = \frac{{\sum }_{i} {N}_{i} {M}_{i}^{2}}{{\sum }_{i} {N}_{i} {M}_{i}}$$

where Nᵢ is the number of molecules of molecular weight Mᵢ as above. The weight average molecular weight can be determined by light scattering, small angle neutron scattering (SANS), X-ray scattering, gel permeation chromatography, and sedimentation velocity.

A copolymer formed upon reaction of a poly(ethylene glycol) with a pyridine-2-thiol-containing monomer, e.g., poly[(2-(pyridin-2-yldisulfanyl)ethyl acrylate-co-[poly(ethylene glycol) (PDA-PEG), can be a precursor copolymer that can be further reacted directly with a compound or monomer containing a functional group of use in the final delivery system (e.g., an antibody, a targeting ligand, a BBB receptor ligand) to form a copolymer that is then crosslinked to form a nanogel. In some embodiments, a copolymer formed upon initial reaction of a poly(ethylene glycol) with a pyridine-2-thiol-containing monomer can be an intermediate copolymer that can then be further reacted to form one or more further intermediates and/or a precursor copolymer that can then be reacted directly with a material for use in the final delivery system and thereby form a copolymer that is then crosslinked to form a nanogel. In general, further functionalization of a precursor or intermediate copolymer can be carried out via reaction of pyridine groups (or reaction products of pyridine groups) of the copolymer.

To conjugate an antibody of interest to a precursor copolymer, pyridine groups of an intermediate copolymer can be activated with a functional group that is conducive to antibody conjugation. For instance, an intermediate copolymer can be functionalized by reaction with an activator having a general structure of Y-Q-X. X and Y independently may be a leaving group, one of which (Y) being capable of reacting with a pyridine group or an intermediary reaction product group provided upon reaction of a monomer with a pyridine group and the other (X) being capable of conjugation with an antibody.

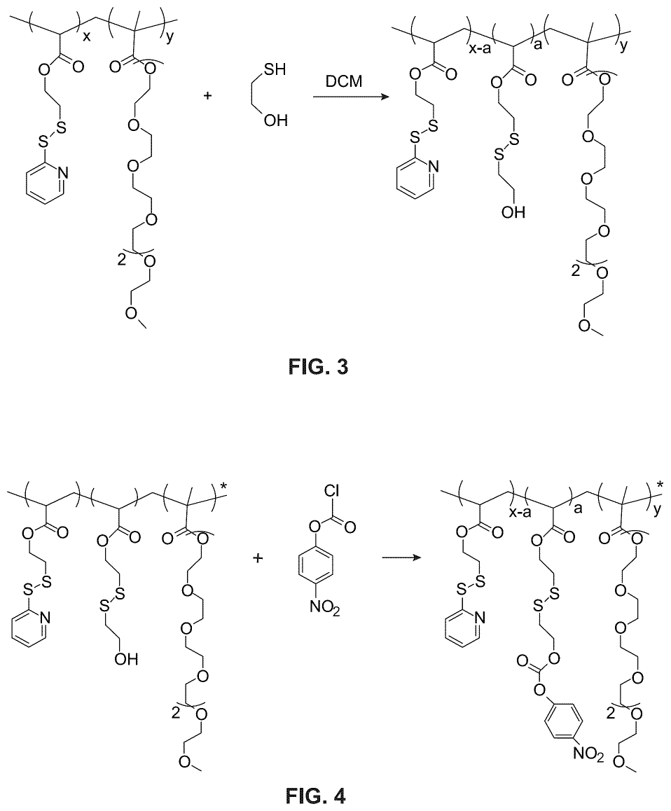

In one embodiment as illustrated in FIG. 3, a first intermediate copolymer formed by reaction of a poly(ethylene glycol) with a pyridine-2-thiol-containing monomer can be modified by reaction of a portion of the pyridine-terminated groups to form a second intermediate copolymer that includes hydroxyl-terminated groups. In further reaction of this second intermediate copolymer (e.g., as shown in FIG. 4), pendant hydroxyl-terminated groups can be reacted with the Y functionality of an activator (e.g., the halogen group of the activator in FIG. 4), with the activated pendant group of the resulting precursor copolymer being capable of conjugation with an antibody via the remaining X functionality (e.g., the nitrophenyl group of the activator in FIG. 4). In this embodiment, the poly(ethylene glycol) component of the first and second intermediate copolymers will generally not terminate in a hydroxyl group, so as to avoid activation of the poly(ethylene glycol) component by reaction with the activator group and to ensure that the activated pendant group is developed from a pyridine-terminated group of the initially formed copolymer.

Examples of materials that can react with a pyridine group to form a hydroxyl-terminated pendant group can include a mercapto alcohol, e.g., 2-mercaptoethanol (BME), 3-mercapto-1-butanol, etc., or other suitable reagents that can react via a thiol-disulfide exchange reaction to provide a terminal hydroxyl group (see, e.g., FIG. 3).

The terminal hydroxyl groups of the intermediate copolymer thus formed can be available for reaction with an activator to provide a precursor copolymer that includes a pendant leaving group, X, suitable for conjugation with an antibody. By way of example, X and Y may include, without limitation, a halide group, a mesyl group, a tosyl group, an aroxyl group such as a phenoxyl group, and a substituted aroxyl group such as a substituted phenoxyl group. Examples of activators include, but are not limited to, chloroformates, e.g., 4-nitrophenyl chloroformate, pentafluorophenyl chloroformate, succinimidyl chloroformate, or combinations thereof.

Following activation, a precursor copolymer can be conjugated to an antibody through reaction of the activated pendant group with the antibody under conditions as are known to those in the art (e.g., pH 8.5, etc.).

Antibodies conjugated to a copolymer can be configured for a therapeutic use following delivery across a blood brain barrier. In one embodiment, an anti-PTB-P1 antibody can be conjugated to a precursor copolymer. Anti-PTB-P1 can intracellularly degrade the PTB-P1 protein in glial cells (e.g., microglia, astrocytes, ependymal cells, Schwann cells, satellite cells, oligodendrocyte lineage cells) and can encourage conversion of the glial cells to neurons. Other antibodies that can be delivered across a blood brain barrier by use of the disclosed nanogels and that can, in some embodiments, degrade the targeted protein can include, without limitation, anti-Amyloid-β (Aβ) antibody, anti-DEAD-box RNA helicase 3 (DDX3) antibody, anti-CD33 antibody, anti-tau antibody, anti-programmed death-ligand 1 (PD-L1), anti-signal transducer and activator of transcription 3 (STAT3), anti-α-synuclein, anti-bone morphogenetic protein-4 (BMP4), anti-OMA1, anti-inositol polyphosphate-5-phosphatase (INPP5D), anti-leucine-rich repeat kinase 2 (LRRK2), anti-stearoyl-CoA desaturase 5 (SCD5), anti-nucleotide-binding domain leucine-rich repeat and pyrin domain containing receptor protein 3 (NLRP3), anti-superoxide dismutase 1 (SOD1), anti-TAR DNA-binding protein 43 (TDP-43), anti-ε4 allele of apolipoprotein E (APOE4), anti-C—C motif chemokine receptor 5 (CCR5), or any combination thereof.

As indicated in FIG. 6, an antibody can be conjugated to a copolymer via a pendant group that includes both a disulfide linkage and an ester linkage. The linkage of the conjugation can be utilized to control degradation of the linkage and delivery of the antibody from a nanogel. An ester bond can be considered an acid-sensitive bond, i.e., a bond that can degrade in an environment of about pH 6.8 or less, for instance, about pH 4 to about pH 6.8, and that can be more stable in an environment at higher pH (e.g., about 7 or higher). A disulfide bond can be considered to be a redox potential sensitive bond, i.e., a bond that can degrade in an environment having a redox potential equal to that of a glutathione concentration of from about 0.1 mM to about 10 mM. The presence of these linkages can serve to maintain the antibody conjugated within the crosslinked nanogel until after passage across the BBB, as the luminal (blood-facing) side of the BBB is generally higher in pH and lower in redox potential than the abluminal (brain-facing) side, which generally exhibits a pH of about 6.5 and a glutathion concentration of about 2.7 mM.

In addition to a copolymer that includes an antibody conjugated thereto, a nanogel can include a ligand for targeting a component on the brain side of the BBB. In one particular embodiment, a targeting ligand can include a phosphorylcholine group.

In one embodiment, a phosphorylcholine group can function as a ligand for a nicotinic acetylcholine receptor (nAchR). nAchR are ligand-gated cation channels located on presynaptic compartment sites where they modulate acetylcholine or other neurotransmitter release, as well as cell excitability and neuronal integration. nAchR expression is extremely broad across the brain, and nAchR have been mentioned as a potential target for multiple therapeutic approaches, for instance antidepressant treatments as well as treatments involving anti-inflammatory pathways and intracellular signaling.

A phosphorylcholine group can function as a ligand for any neuronal subtype nAchR present on the abluminal side of the BBB or on the BBB itself, including both homomeric and heteromeric combinations of the twelve different nicotinic receptor subunits: α2-α10 and β2-β4. Examples of nAchR neuronal subtypes that can be targeted by a phosphorylcholine ligand of a nanogel can include, without limitation, (α₄)₃(β₂)₂, (α₄)₂(β₂)₃, (α₃)₂(β₄)₃, α₄α₆β₃(β₂)₂, and (α₇)₅, as well as combinations thereof. In one particular embodiment, a phosphorylcholine ligand can target an α₇ nAchR that includes only α₇ subunits. In one embodiment, a phosphorylcholine ligand can target nAchR as are expressed by glial cells or endothelial cells, for instance, in therapeutic delivery of an anti-PTB-P1 antibody to the interior of the glial cells and subsequent conversion of the glial cells to neurons. A phosphorylcholine group can also facilitate crossing of the BBB, for instance, in conjunction with a ligand for a BBB receptor as described further herein.

In formation of a copolymer for use in formation a nanogel, a precursor copolymer (e.g., a PDA-PEG copolymer as initially formed) can be reacted with a monomer that includes a phosphorylcholine group. Alternatively, a PDA-PEG copolymer can be an intermediate copolymer that can be further modified to form a precursor copolymer that includes suitable functionality for reaction with a monomer that includes a phosphorylcholine groups.

In one embodiment, a phosphorylcholine group-containing monomer can include the general structure of:

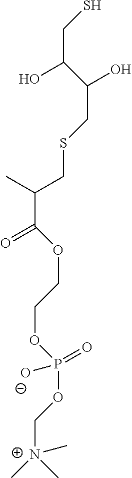 [SMILES: CC(CSCC(O)C(O)CS)C(=O)OCCOP(=O)([O-])OC[N+](C)(C)C]

in which the phosphorylcholine head of the monomer includes only a single carbon between the ammonium group and the phosphate group, rather than a two-carbon ethyl group as is more common in phosphoryl choline monomers. As described further in the Examples section below, utilization of a monomer including such a phosphorylcholine group can provide for formation of a nanogel with desirable characteristics for delivery of a nanogel in treatment of a neurodegenerative or retinal degenerative disease.

Of course, a phosphorylcholine-containing monomer is not limited to such an embodiment. Examples of a monomer including a phosphorylcholine group can include, without limitation, 2-acryloyloxyethyl phosphorylcholine, 2-methacryloyloxyethyl phosphorylcholine (MPC), 2-(meth)acryloyloxyethoxyethyl phosphorylcholine, 6-(meth)acryloyloxyhexyl phosphorylcholine, 10-(meth)acryloyloxyethoxynonyl phosphorylcholine, allyl phosphorylcholine, butenyl phosphorylcholine, hexenyl phosphorylcholine, octenyl phosphorylcholine, decenyl phosphorylcholine, or combinations thereof.

One or more copolymers of the crosslinked nanogels can be conjugated with a ligand for a BBB receptor. A suitable BBB receptor ligand can be a small molecule or polypeptide that can facilitate transportation of the nanogels across the BBB to deliver the antibody to the brain. By way of example, and without limitation, a ligand for a BBB receptor can include a ligand for one or more of scopine receptor, glutathione receptor, transferrin receptor, melanotransferrin receptor, adenosine receptor, insulin receptor, low-density lipoprotein receptor, leptin receptor, thiamine receptor, rabies virus glycoprotein receptor, TAT peptide receptor, encephalin receptor, angiopep-2 receptor, diphtheria toxin receptor, receptor for advanced glycation end-products (RAGE), tetanus toxin receptor, or any combination thereof.

A ligand for a BBB receptor can be conjugated to a copolymer either prior to or following crosslinking of the copolymer(s) to form the nanogels. A BBB receptor ligand can be conjugated to a polymer via remaining functionality of the copolymer, e.g., remaining pyridine functionality or remaining intermediate activator functionality. For instance, a compound that includes a BBB receptor ligand and a free thiol group (e.g., certain peptide or small molecule ligands) can conjugate to a copolymer or nanogel through a thiol-disulfide exchange reaction of pyridine-terminated groups of the copolymer. In some embodiments, a BBB receptor ligand can be conjugated to a copolymer through reaction with unreacted activator leaving groups. In other embodiments, pyridine-terminated groups of one or both copolymers can be functionalized prior to or following crosslinking to include a functional group configured to react with a compound including a ligand for a BBB receptor. In one embodiment, conjugation of a BBB receptor ligand can be carried out following crosslinking, so as to ensure a high concentration of the BBB receptor ligand at the surface of the nanogels.

The particle form of the delivery system can be provided via crosslinking of one or more of the functionalized copolymers. For instance, a PDA-PEG-based copolymer can be subjected to disulfide bond cleavage of remaining pyridine-terminated groups followed by oxidation to crosslink the polymers and form a nanoparticle. Due to the relatively large size of an antibody in general, upon crosslinking, the nanogels can encapsulate the antibody payload within the nanogel. Other smaller end groups of the pendant groups can be present both on the surface and within the interior of the nanogel. For instance, hydrophilic groups including the poly(ethylene glycol) pendant groups and phosphorylcholine-containing pendant groups can exhibit high concentration at the nanogel surface. As mentioned previously, to ensure high concentration of a ligand for a BBB receptor on the nanogel surface, in one embodiment, a compound including a ligand for a BBB receptor can be conjugated to the nanogel following crosslinking.

Beneficially, disclosed nanogels can be utilized to deliver an antibody across the BBB by use of systemic delivery. As utilized herein, “systemic delivery” refers to delivery that leads to a broad biodistribution of a compound within an organism. For instance, a composition including a nanogel as described can be delivered to a subject in need thereof via systemic enteral or parenteral delivery, e.g., via injection, infusion, or implantation.

A composition can include additional agents in addition to the nanogels. Such agents can be active agents providing direct benefit to a tissue, or they may be supporting agents, improving delivery, compatibility, or reactivity of other agents in the composition.

Compositions for systemic deliver can include pharmaceutically acceptable sterile aqueous or nonaqueous solutions, dispersions, suspensions, or emulsions as well as sterile powders for reconstitution into sterile injectable solutions or dispersions just prior to use. Examples of suitable aqueous and nonaqueous carriers, diluents, solvents, or vehicles include water, ethanol, polyols (e.g., glycerol, propylene glycol, polyethylene glycol and the like), carboxymethylcellulose and suitable mixtures thereof, vegetable oils (e.g., olive oil), and injectable organic esters such as ethyl oleate. In addition, the composition can contain minor amounts of auxiliary substances such as wetting or emulsifying agents, pH buffering agents and the like that can enhance the effectiveness of the cannabidiol. Proper fluidity may be maintained, for example, by the use of coating materials such as lecithin, by the maintenance of the required particle size in the case of dispersions, and by the use of surfactants. Compositions may also contain adjuvants such as preservatives, wetting agents, emulsifying agents, and dispersing agents.

Prevention of the action of microorganisms may be ensured by the inclusion in a composition of various antibacterial and antifungal agents such as paraben, chlorobutanol, phenol, sorbic acid, and the like. It may also be desirable to include isotonic agents such as sugars, sodium chloride, and the like.

A composition can include one or more buffers as are generally known in the art. For example, a composition may be formulated with inclusion of a biocompatible buffer such as distilled water, saline, phosphate buffers, borate buffers, HEPES, PIPES, and MOPSO. In one embodiment, a composition may be formulated to have a pH of between about 6.6 and about 7.4.

In one embodiment, a composition can be delivered intravenously in a systemic delivery protocol. For example, osmotic mini pumps may be used to provide controlled delivery of high concentrations of the nanogel through cannulae to a site for systemic delivery, such as directly into a blood vessel.

Following systemic delivery, the nanogels can circulate to the BBB where they can cross the BBB and deliver the antibodies encapsulated therein to the abluminal side of the BBB; for instance, following uptake by cells on the brain side of the BBB. As previously stated, the nanoparticle delivery system can be responsive to acidic pH and/or high glutathione (GSH) environment to release the antibody payload. The nanoparticle delivery system can thus provide an ideal tool for brain-targeted delivery.

The present disclosure may be better understood with reference to the Examples set forth below.

### Example 1

A PDA-PEG-OCH₃ polymer was synthesized through free radical polymerization (FIG. 1). Briefly, 800 mg (3.32 mmol) 2-(pyridin-2-yldisulfanyl)ethyl acrylate (PDSA) and 1.69 g (3.32 mmol) poly(ethylene glycol) methyl ether methacrylate (PEGMMA) were dissolved in 15 mL anisole in a 50 mL round bottom flask and degassed with nitrogen for 30 minutes at room temperature. Following, a degassed solution of azobisisobutyronitrile (AIBN) (54.22 mg, 0.33 mmol) in 2 mL anisole was added dropwise into the reaction mixture. The flask was then immersed in an oil bath maintained at 68° C. and stirred for 24 hours in the dark. Following the reactions, the resulted polymers were collected by precipitation with ice-cold diethyl ether. For further purification, the collected polymers were dissolved in DCM and then precipitated with ice-cold diethyl ether for five times. The purified polymers were dried under vacuum in the dark until the solvents were completely removed. The structural composition of PDA-PEG-OCH₃ was analyzed by 1H NMR using CDCl₃ as the solvent. The yield of PDA-PEG-OCH₃ was 99% (1.683 g).

A PDA-PEG-OH polymer was synthesized through free radical polymerization (FIG. 2). In brief, 500 mg (3.81 mmol) PDSA and 1.34 g (3.81 mmol) PEGMA-OH were dissolved into 15 mL anisole in a 50 mL round bottom flask and degassed with nitrogen for 30 minutes at room temperature. Following, a degassed solution of AIBN (62.58 mg, 0.381 mmol) in 2 mL anisole was added dropwise into the reaction mixture. The flask was then immersed in an oil bath maintained at 68° C. and stirred for 24 hours in the dark. Following the reactions, the resulting polymers were collected by precipitation with ice-cold diethyl ether. For further purification, the collected polymers were dissolved in DCM and then precipitated with ice-cold diethyl ether for five times. The purified polymers were dried under vacuum in the dark until the solvents were completely removed. The structural composition of PDA-PEG-OH was analyzed by 1H NMR using CDCl₃ as the solvent. The yield of PDA-PEG-OH was 94% (2.166 g).

An intermediate polymer PDA-PEG-BME was prepared via a thiol-disulfide exchange reaction between 2-mercaptoethanol (BME) and polymer PDA-PEG-OCH₃ (FIG. 3). PDA-PEG-OCH₃, as described above (609 mg, 841.29 μmol), was dissolved in 10 mL dichloromethane (DCM) with a catalytic amount of glacial acetic acid. While vigorously stirring, BME (39.44 mg, 504.77 μmol) in 500 μL DCM was added dropwise. The reaction was kept stirring overnight in the dark at room temperature. The targeted product was then precipitated with ice-cold diethyl ether and further purified through precipitation with DCM and ice-cold diethyl ether for five times. The polymer PDA-PEG-BME was dried under vacuum in the dark for 48 hours (572 mg, 72%), and its structural composition was confirmed by 1H NMR.

PDA-PEG-BME (572 mg) was dissolved in 5 mL DCM supplemented with 38 μL pyridine. While vigorously stirring at 4° C., 4-nitrophenyl chloroformate (NPC) (133.55 mg, 664 μmol) in 500 μL DCM was added dropwise as shown in FIG. 4. The reaction solution was stirred at 4° C. in the dark for 24 hours. The desired product PDA-PEG-NPC was precipitated with ice-cold diethyl ether and further purified through precipitation with DCM and ice-cold diethyl ether for five times. The PDA-PEG-NPC polymer was dried under vacuum in the dark for 48 hours (482 mg, 71.7%), and its structural composition was confirmed by 1H NMR.

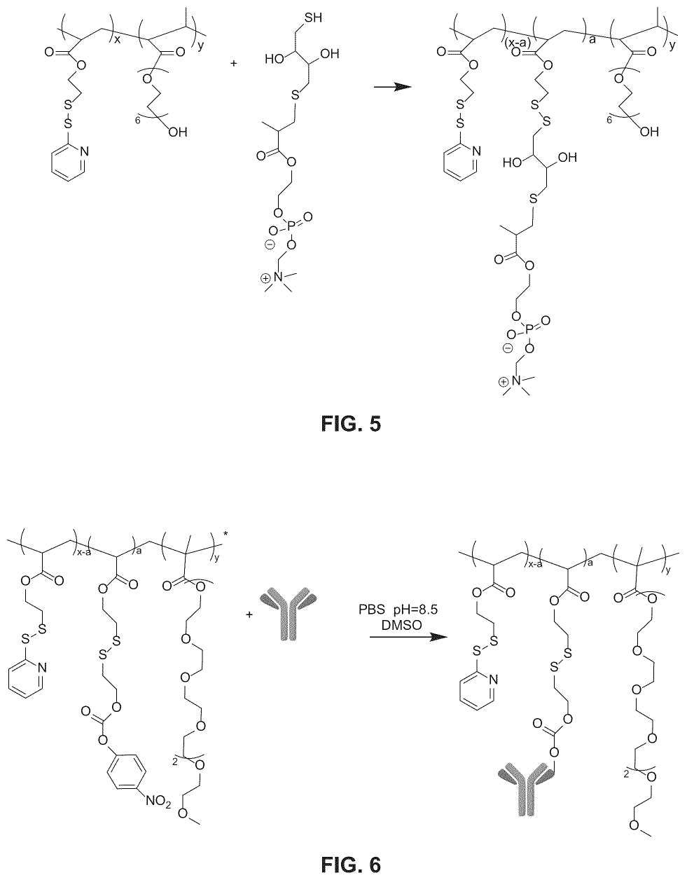

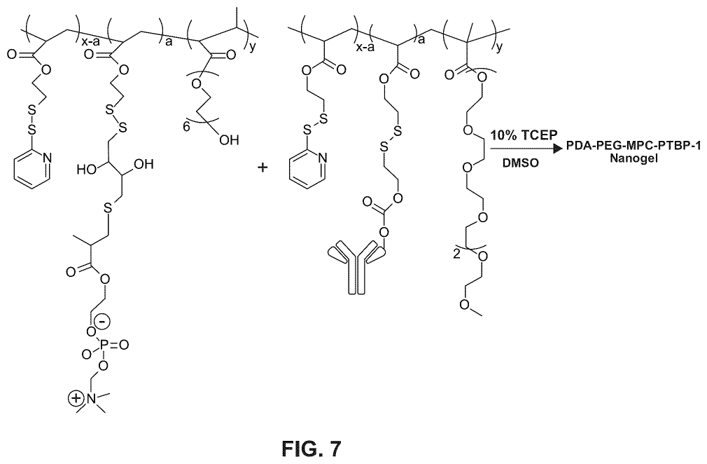

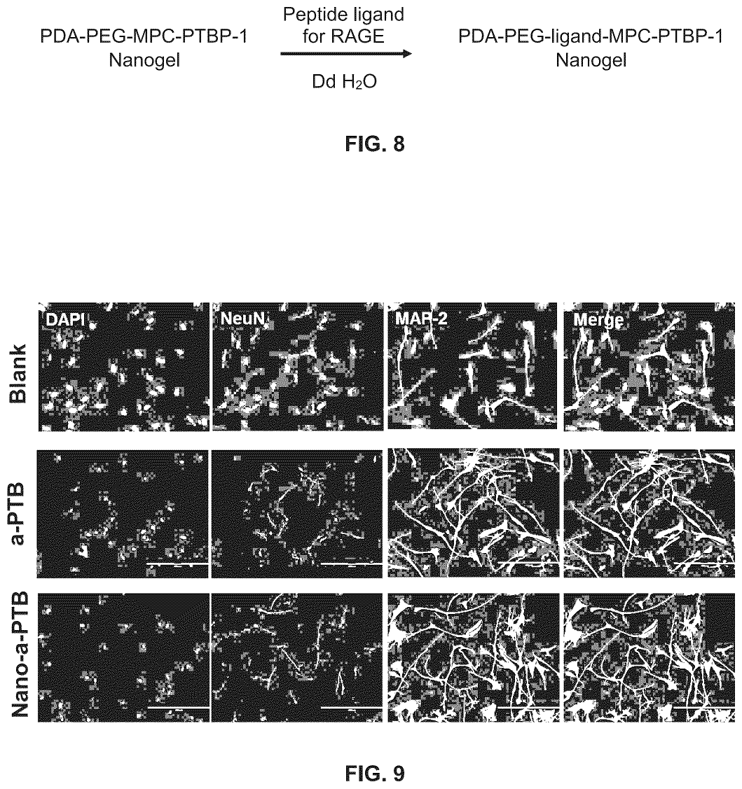

To form the phosphorylcholine-containing precursor, PDA-PEG-OH 100 mg (160.82 μmol) was dissolved in 5 mL of dimethyl sulfoxide (DMSO). Glutathione-2-Methacryloyloxyethyl phosphorylcholine conjugate (GSH-MPC) (35.02 mg, 80.41 μmol) in 500 μL DMSO was added dropwise to the solution under stirring as shown in FIG. 5. The reaction was kept under dark for 24 hours. The reaction mixture was dialyzed against DMSO and then precipitated with ice-cold diethyl ether. The polymer precursor product PDA-PEG-MPC was dried under vacuum in the dark for 48 hours (151 mg, 79%), and its structural composition was confirmed by 1H NMR.

An antibody polymer conjugate (PDA-PEG-aPTB) was formed as follows: To a pre-cooled solution of 45 mg polymer PDA-PEG-NPC in 0.3 mL DMSO, anti-polypyrimidine tract binding protein 1 (anti-PTB-P1) (0.45 mg in 0.7 mL PBS, pH=8.5) was added dropwise at 4° C. under vigorous stirring, and the resulting solution was stirred for 48 hours at 4° C. in the dark as shown in FIG. 6. The process of conjugation was monitored by measuring the absorbance of released side product 4-nitrophenol at 400 nm using UV-Vis spectroscopy. When the reaction was complete, the reaction mixture was purified through dialysis in Spectra/Por® dialysis tube (regenerated cellulose, MWCO: 100 kDa) against PBS buffer for 48 hours at 4° C.

To form a nanogel, PDA-PEG-MPC precursor (500 mg) was dissolved in 4 mL of DMSO. Following, PDA-PEG-aPTB precursor (45 mg in 0.5 mL PBS) was added dropwise at room temperature under stirring. Five minutes later, 25 mg (tris(2-carboxyethyl)phosphine (TCEP) dissolved in 8 mL of DMSO was added dropwise under stirring. The mixture was kept at room temperature for 15 minutes to initiate the crosslinking process. The mixture solution was then added dropwise into 50 mL double distilled H₂O at 4° C. under stirring. The produced nanogels were purified through dialysis in Spectra/Por® dialysis tube (regenerated cellulose, MWCO: 100 kDa) against PBS buffer for 48 hours at 4° C. The final nanogels were stored in PBS (pH 7.4) at 4° C. for use.

A portion of the nanogels were decorated with a peptide ligand (CKLVFFAED, SEQ ID NO: 1) for the receptor for advanced glycation end product peptide (RAGE) following nanogel formation to provide dual response nanogels. To a PDA-PEG-MPC-aPTBP1 nanogel dispersion in PBS buffer (pH 7.4) was added a solution comprising the peptide ligand (1 mg/mL) in DMSO and this was then stirred overnight at 4° C. The receptor ligand-modified nanogels were purified through dialysis in Spectra/Por® dialysis tube (MWCO: 8 kDa) against PBS buffer for 48 hours at 4° C. The particle size, size distribution, and zeta potential of the nanogels were determined by dynamic light scattering (DLS), recorded on ZETASIZER® Nano ZS (Malvern Instruments Ltd., Malvern, UK).

### Example 2

An in vitro BBB Transwell® model was constructed following methods previously described in the literature. Briefly, hCMEC/D3 cells were seed on a polycarbonate 24-well Transwell® membrane of 1 μm mean pore size precoated with collagen at a density of 10,000 cells/well. The transendothelial electrical resistance (TEER) of cell monolayers was monitored every day by using an epithelial voltohmmeter (Millicell®-RES, Millipore, USA). The BBB model could be used when the TER was above 180 Ω/cm².

Converting human astrocytes to neurons was carried out using the in vitro BBB model. Glass coverslips were coated with poly-lysine overnight, washed with sterile ultrapure water twice, and then placed into the wells of 24-well plates. After that, human astrocyte (HA) cells were seeded at 20,000/well per well onto the coverslips in HA culture medium. After overnight incubation, the old medium was replaced with F12 medium. The Transwell® inserts, prepared as described above, were added to the top of the 24-well plates with HA cells in culture. Different treatments, either blank, free PTBP-1 antibody (a-PTB) (20 μg/mL), or Nano-a-PTB nanogels, as described above (20 μg/mL), were added to the top chamber and replaced with fresh treatments every 2 days for two replacements. Six days later, the coverslips were washed with PBS twice and fixed with 4% paraformaldehyde for 20 minutes at room temperature. The cells were washed with PBS twice. The expression of NeuN and MAP-2 were detected with anti-NeuN and anti-MAP-2 antibodies and detected with anti-rabbit secondary antibody. The slides were observed by the ZEISS® LSM700 (Carl Zeiss) confocal microscope.

After the treatment of PTBP-1 antibody (a-PTB) and Nano-a-PTB, the success of converting human astrocytes to neurons was evidenced by the positive staining for NeuN and MAP-2 as shown in FIG. 9.

### Example 3

AD transgenic mice (5XFAD) at the age of 6 months old were randomly divided into three groups and intravenously injected with either PBS, free a-PTB, or Nano-a-PTB at the a-PTB equivalent dose of 1.6 mg/kg by intravenous injection once per week for two weeks. Normal C57 BL/6 mice (WT-like littermates) at the same age were given saline and used as the Normal group.

An assessment of nest construction capacity was performed once a week in the duration of treatment referring to a published protocol. In brief, each mouse was caged and housed individually, approximately 1 hour before the dark phase, one pressed cotton square (5×5 cm, Nestlets™, Ancare, Bellmor, USA) was placed in the home cage. Next morning, the nest was photographed and scored as follows:
1=not noticeably touched,
2=partially torn up,
3=mostly shredded but often no identifiable site,
4=identifiable but flat,
5=perfect or nearly so.

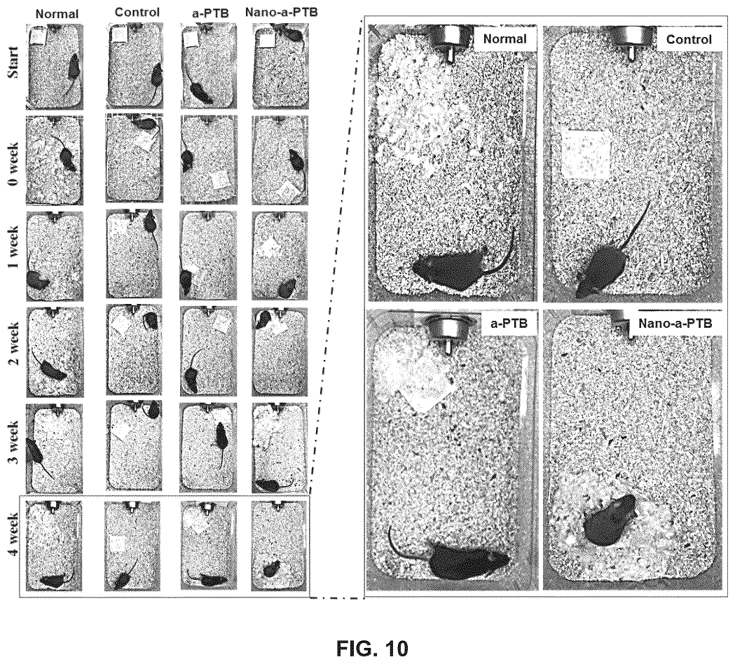

The nest construction assay revealed that after two doses of treatment, the AD mice in the a-PTB treatment group partially recovered their nesting skills (FIG. 10). As shown in FIG. 10, the Nano-a-PTB treatment yielded a fully recovered nesting skill for AD mice.

### Example 4

To evaluate the spatial learning and memory ability of mice following different treatments, a Morris water maze (MWM) test was carried out on groups of mice as described in Example 3.

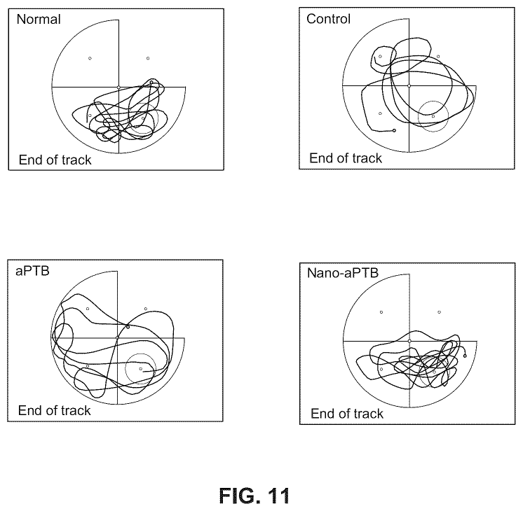

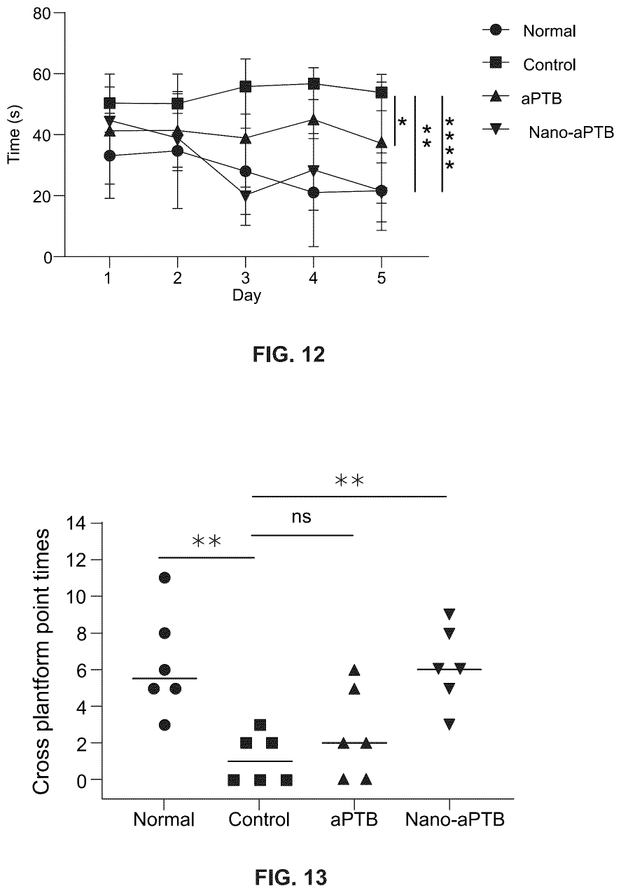

The performance of the mice was recorded through videos and analyzed by EthoVision® XT15 software to determine the path length, latency to reach the platform, and swim speed for each mouse during the test. The number of targeted platform crossings for each animal during the 60 seconds was recorded. After two weeks of treatment, Nano-aPTB-treated AD mice spent most of the time in the targeted quadrant (FIG. 11), which was different from that shown in control and aPTB-treated AD mice while similar to that shown in the normal mice. Furthermore, Nano-aPTB-treated AD mice showed similar learning capacity as the normal mice (FIG. 12), evidenced by the declined escape latency time during the training session, while no significant change was observed in control and aPTB-treated AD groups. In addition, the increased number of crossing the targeted platform for the Nano-aPTB-treated AD mice suggested their improved memory ability as compared to that of the control and aPTB groups (FIG. 13).

While certain embodiments of the disclosed subject matter have been described using specific terms, such description is for illustrative purposes only, and it is to be understood that changes and variations may be made without departing from the spirit or scope of the subject matter.

1. A method of treatment for a neurodegenerative or retinal disorder, comprising:
systemically delivering a composition to a subject, the composition comprising a nanogel that includes a crosslinked network comprising a first copolymer and a second copolymer, the first copolymer including a first backbone, a first group pendant to the first backbone comprising a first poly(ethylene glycol), and a second group pendant to the first backbone that is conjugated to an antibody, the second copolymer including a second backbone, a third group pendant to the second backbone comprising a second poly(ethylene glycol), and a fourth group pendant to the second backbone comprising a phosphorylcholine group, the nanogel comprising a ligand for a blood brain barrier receptor at a surface of the nanogel; wherein
following the system delivery, the nanogel crosses the blood brain barrier and the second group is degraded, thereby releasing the antibody from the nanogel following the crossing of the blood brain barrier; and wherein
the antibody targets an antigen and degrades the antigen in treatment of the neurodegenerative or retinal disorder.

2. The method of claim 1, wherein the antibody comprises an anti-polypyrimidine tract binding protein 1, an anti-Amyloid-β, an anti-DEAD-box RNA helicase 3, an anti-CD33, an anti-tau, an anti-Programmed death-ligand 1, an anti-Signal Transducer and Activator of Transcription 3, an anti-α-synuclein, an anti-Bone morphogenetic protein-4, an anti-OMA1, an anti-inositol polyphosphate-5-phosphatase, an anti-leucine-rich repeat kinase 2, an anti-Stearoyl-CoA desaturase 5, an anti-nucleotide-binding domain leucine-rich repeat and pyrin domain containing receptor protein 3, an anti-superoxide dismutase 1, an anti-TAR DNA-binding protein 43), an anti-ε4 allele of apolipoprotein E, an anti-C—C Motif Chemokine Receptor 5, or a combination thereof.

3. The method of claim 1, wherein the neurodegenerative or retinal disorder comprises Alzheimer's disease, Amyotrophic lateral sclerosis, Friedreich's ataxia, Huntington's disease, Lewy body disease, Parkinson's disease, spinal muscular atrophy, multiple sclerosis, neonatal hypoxic-ischemic, stroke, spinal cord injury, brain injury, retina injury, age-related macular degeneration, myopic related macular degeneration, post-traumatic stress disorder, or frontotemporal dementia.

4. The method of claim 1, wherein the fourth group comprises a reaction product of a phosphorylcholine monomer comprising the following structure:

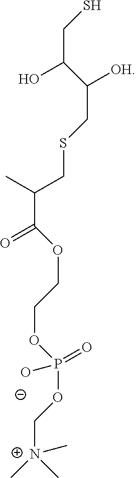 [SMILES: CC(CSCC(O)C(O)CS)C(=O)OCCOP(=O)(O)OC[N+](C)(C)C]

5. The method of claim 1, wherein the phosphorylcholine group is a targeting ligand for a nicotinic acetylcholine receptor expressed on an endothelial cell or a glial cell, wherein the antibody is released from the nanogel within the endothelial cell or the glial cell.

6. The method of claim 5, wherein the glial cell is a microglia, an astrocyte, an ependymal cell, a Schwann cell, a satellite cell, an oligodendrocyte lineage cell, or a combination thereof.

7. The method of claim 6, wherein the antibody comprises an anti-polypyrimidine tract binding protein 1 antibody, and the antibody induces the degradation of polypyrimidine tract binding protein 1 and induces conversion of the glial cell to a neuron.

8. The method of claim 1, wherein the systemic delivery comprises parenteral injection of the composition to the subject.

9. A nanogel comprising:
a first copolymer comprising a first backbone, a first group pendant to the first backbone comprising a first poly(ethylene glycol) and a second group pendant to the first backbone including a conjugation to an antibody;
a second copolymer comprising a second backbone, a third group pendant to the second backbone comprising a second poly(ethylene glycol), and a fourth group pendant to the second backbone comprising a phosphorylcholine group; and
a ligand for a blood brain barrier receptor at a surface of the nanogel; wherein
the first copolymer and the second copolymer are crosslinked to one another.

10. The nanogel of claim 9, wherein the first copolymer further comprises a fifth group pendant to the first backbone that terminates in a pyridine group.

11. The nanogel of claim 9, wherein the second copolymer further comprises a sixth group pendant to the second backbone that terminates in a pyridine group.

12. The nanogel of claim 9, wherein the ligand for a blood brain barrier receptor comprises a ligand for a scopine receptor, glutathione receptor, transferrin receptor, melanotransferrin receptor, adenosine receptor, insulin receptor, low-density lipoprotein receptor, leptin receptor, thiamine receptor, rabies virus glycoprotein receptor, TAT peptide receptor, encephalin receptor, angiopep-2 receptor, diphtheria toxin receptor, receptor for advanced glycation end-products (RAGE), tetanus toxin receptor, or any combination thereof.

13. The nanogel of claim 9, wherein the fourth group comprises a reaction product of a phosphorylcholine monomer comprising the following structure:

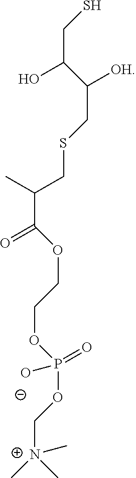 [SMILES: CC(CSCC(O)C(O)CS)C(=O)OCCOP(=O)(O)OC[N+](C)(C)C]

14. A method for forming a nanogel comprising:
conjugating a first precursor copolymer with an antibody to form a first copolymer, the first precursor copolymer comprising a first backbone, a first group pendant to the first backbone that includes a first poly(ethylene glycol), a second group pendant to the first backbone that includes a first functional group configured to conjugate the antibody, and a third group pendant to the first backbone that includes a terminal pyridine;
conjugating a second precursor copolymer with a monomer that includes a phosphorylcholine group to form a second copolymer, the second precursor copolymer comprising a second backbone, a fourth group pendant to the second backbone that includes a second poly(ethylene glycol), a fifth group pendant to the second backbone that includes a second functional group configured to form a bond with the monomer, and a sixth group pendant to the second backbone that includes a terminal pyridine;
crosslinking the first copolymer with the second copolymer; and
conjugating a ligand for a blood brain barrier receptor to the first copolymer and/or to the second copolymer.

15. The method of claim 14, wherein the ligand for the blood brain barrier receptor is conjugated to the first copolymer and/or to the second copolymer following crosslinking of the first copolymer with the second copolymer.

16. The method of claim 14, further comprising forming the first precursor copolymer and the second precursor copolymer.

17. The method of claim 16, wherein the first precursor copolymer is formed according to a process that includes reaction of a third poly(ethylene glycol) with a first pyridine-2-thiol-containing monomer and the second precursor copolymer is formed according to a process that includes reaction of a fourth poly(ethylene glycol) with a second pyridine-2-thiol-containing monomer.

18. The method of claim 17, wherein the first pyridine-2-thiol-containing monomer and the second pyridine-2-thiol-containing monomer are independently selected from (pyridine-2-thiol)ethyl acrylate, (pyridine-2-thiol)ethyl methacrylate, N-(2-(pyridin-2-yldisulfanyl)ethyl) acrylamide, N-(2-(pyridin-2-yldisulfanyl)ethyl)methacrylamide, and ethyl(2-(pyridin-2-yldisulfanyl)ethyl) carbonate.

19. The method of claim 17, wherein reaction of the third poly(ethylene glycol) with a first pyridine-2-thiol-containing monomer forms a first intermediate copolymer, the method further comprising reaction of the first intermediate copolymer with an activator to form a second intermediate copolymer, the second intermediate copolymer comprising a terminal leaving group configured for conjugation with the antibody.

20. The method of claim 19, wherein the activator comprises a chloroformate.

21. The method of claim 14, wherein the second group, the third group, the fifth group, and the sixth group all include a disulfide linkage and an ester linkage on the pendant group.
